(19) United States
(12) Patent Application Publication
(10) Pub. No.: US 2023/0218671 A1
(43) Pub. Date: Jul. 13, 2023

(54) CHIMERIC ANTIGEN RECEPTOR REGENERATIVE GAMMA DELTA T CELLS

(71) Applicant: CREATIVE MEDICAL TECHNOLOGIES, INC., Phoenix, AZ (US)

(72) Inventors: Thomas Ichim, San Diego, CA (US); Amit Patel, Salt Lake City, UT (US); Timothy Warbington, Phoenix, AZ (US)

(73) Assignee: CREATIVE MEDICAL TECHNOLOGIES, INC., Phoenix, AZ (US)

(21) Appl. No.: 18/082,841

(22) Filed: Dec. 16, 2022

Related U.S. Application Data
(60) Provisional application No. 63/297,876, filed on Jan. 10, 2022.

(51) Int. Cl. A61K 35/17; A61P 43/00

(52) U.S. Cl. CPC A61K 35/17; A61P 43/00

(57) ABSTRACT

Disclosed are novel method of inducing tissue regeneration through administration of gamma delta T cells that have been endowed regenerative activity. In one embodiment said regenerative active is bestowed upon said cells by culture with a regenerative cell population. In other embodiments a chimeric antigen receptor (CAR) is transfected which induces generation of regenerative substances upon activation of said CAR. Regenerative factors useful for the treatment of the invention depend on the condition for which treatment is desired, for example, in neurological conditions production of brain derived neurotrophic factor is produced upon activation of said CAR, wherein said CAR recognizes antigens or neoantigens associated with neuronal injury.

### CROSS-REFERENCE TO RELATED APPLICATIONS

This application claims the benefit of priority to U.S. Provisional Application No. 63/297,876, filed Jan. 10, 2022, which is incorporated herein by reference in its entirety.

### FIELD OF THE INVENTION

The teachings herein relate to Chimeric Antigen Receptor Regenerative Gamma delta T lymphocytes and methods of using the same for treating degenerative disorders.

### BACKGROUND

Gamma delta T lymphocytes represent a minor subset of peripheral blood in humans (less than 10%) [1, 2]. These cells have been implicated in numerous pathological situations including malaria [3], cancer, rheumatoid arthritis [4-17], uveitis [18], burn injury [19], Behcet's disease [20], and intrauterine inflammation [21].

Gamma delta T cells expressing V.gamma.9V.delta.2 (gamma 9 delta 2) T cell receptor recognise the endogenous isopentenyl pyrophosphate (IPP) that is over produced in cancer cells as a result of dysregulated mevalonate pathway.

The ability of gamma delta T lymphocytes to produce abundant pro inflammatory cytokines like IFN-gamma, their potent cytotoxic effective function and MHC-independent recognition of antigens makes them an important layer of cancer immunotherapy. Gamma delta T cells have been indicated to be able to kill many different types of tumour cell lines and tumour in vitro, including leukaemia, neuroblastoma, and various carcinomas. Further, it has been demonstrated that gamma delta T cells can recognise and kill many different differentiated tumor cells either spontaneously or after treatment with different bisphosphonates, including zoledronate. Human tumour cells can efficiently present aminobisphosphonate and pyrophosphomonoester compounds to gamma delta T cells inducing their proliferation and IFN-gamma production.

Autologous transplantation strategies of gamma delta T cells have been utilised to overcome the disadvantages associated with allogeneic stem cell transplantation. As part of such autologous transplantation techniques, methods of inducing and culturing sufficient numbers of gamma delta T cells for exerting therapeutic effect autologously have been previously disclosed, for example US 2002/0107392. However, autologous treatment strategies suffer from a number of disadvantages.

Clinical trials employing CAR expressing T cells (CAR-T) have demonstrated the CAR-T approach and in 2014 anti-CD19 CART cell therapy was approved by the United States' FDA. Whilst impressive response rates have been observed in CAR-T trials, currently the technology is somewhat limited by a lack of true disease antigens i.e. antigens expressed only in disease state cells and not in healthy cells. To date, the vast majority of CAR-T therapies have been CD19 targeted. CD19 is expressed in B cell malignancies but it is also expressed on healthy B cells. In this context, CD19-targeted CAR-T treatment can be tolerated, however patients are suffering an increased risk of infections as their immune system is significantly compromised. This is not the case for the majority of other tumour types, specifically solid tumours, where the targeting of healthy tissue would be intolerable. A further limitation of CAR-T therapies trialed clinically to date, is the occurrence of relapse due to the development of resistance to the CAR-T therapy. This has been observed in clinical trials of CD19-targeting CAR-T therapy and is achieved by the emergence of cancer cells which exhibit alternate splicing and/or deleterious mutations of the target (CD19) gene. These ‘escape variants’ result in modified target (CD19) protein which is unrecognizable by the scFv portion of the CAR (whilst retaining sufficient of functionality of the gene). This is a limitation of any single targeting approach; in the context of proliferating cells i.e. cancer, this provides a positive selective pressure to abrogate target antigen expression. Unfortunately the use of CAR-T has almost always been limited to the field of oncology. The use of antigen-specific T cells to selectively induce non-immunological activities is of interest to various fields of medicine. For example, it is known that T cells are actually involved in the process of placentation. With placentation requiring angiogenesis. The invention teaches, in one embodiment, the utilization of T cells to selectively induce angiogenesis in conditions that may benefit, such as ischemic conditions.

In the current application we utilize the gamma delta T cells as a population of cells useful for regenerative purposes through conditioning with regenerative cells or derivatives thereof, or alternatively gamma delta T cells may be transfected with CARs or TCRs to induce expression of regenerative genes.

### SUMMARY

Claims

Preferred embodiments are directed to method of treating a degenerative condition comprising: a) extracting a cellular population resembling gamma delta T cells; b) expanding said population ex vivo; c) endowing said cell with one or more regenerative activities; and d) administering said cell in a patient in need of therapy.

Preferred method include embodiments wherein said T cells express CD3.

Preferred method include embodiments wherein said T cells express CD6.

Preferred method include embodiments wherein said T cells express CD27.

Preferred method include embodiments wherein said T cells express il-2 receptor.

Preferred method include embodiments wherein said T cells express CD25.

Preferred method include embodiments wherein said T cells proliferate in response to IL-2.

Preferred method include embodiments wherein said T cells proliferate in response to IL-7.

Preferred method include embodiments wherein said T cells express the gamma delta T cell receptor.

Preferred method include embodiments wherein said T cells do not express the alpha beta T cell receptor.

Preferred method include embodiments wherein said T cells recognized conserved antigens.

Preferred method include embodiments wherein said T cells are less immunogenic as compared to conventional T cells.

Preferred method include embodiments wherein said conventional T cells are CD4 alpha beta T cells.

Preferred method include embodiments wherein said conventional T cells are CD8 alpha beta T cells.

Preferred method include embodiments wherein said immunogenicity means ability to stimulate proliferation of allogeneic T cells.

Preferred method include embodiments wherein said immunogenicity means ability to stimulate cytotoxicity of allogeneic T cells.

Preferred method include embodiments wherein said immunogenicity means ability to stimulate NF-kappa B activation in allogeneic T cells.

Preferred method include embodiments wherein said immunogenicity means ability to stimulate cytokine secretion of allogeneic T cells.

Preferred method include embodiments wherein said cytokine is IL-2.

Preferred method include embodiments wherein said cytokine is IL-4.

Preferred method include embodiments wherein said cytokine is IL-7.

### DETAILED DESCRIPTION OF THE INVENTION

The current patent application relates to methods of preparing and using gamma delta T cells (.gamma..delta.T cells) as regenerative cells for treatment of conditions associated with tissue degradation and/or degeneration. Said cells suitably the use of gamma delta T cells in allogeneic or autologous recipient subjects and said cells make use of chimeric antigen receptors (CARs) which induce activation regenerative compounds such as cytokines, growth factors or peptides. The invention also relates to processes for the generation of .gamma..delta.T cells expressing chimeric antigen receptors and for detecting CAR expression or TCRs in an antigen specific manner, wherein said antigen relates to injury associated antigens. In addition, the present invention relates to the pharmaceutical use of the cells generated with the processes discussed herein in the treatment of diseases such as degenerative diseases.

In some embodiments of the invention gamma delta T cells are generated from peripheral blood. For example, in one embodiment, said gamma delta T cells are extracted. Suitably, in embodiments .gamma..delta.T cells of the Vgamma9 subtype may be selectively expanded from PBMCs or cord blood mononuclear cells (CBMCs) [22-26]

Generation of cells of the invention may be accomplished by deriving cells from peripheral blood or tissue derived cells in a chemically defined culture medium, including IL-2, serum/plasma and activation by the provision of an aminobisphosphonate such as zoledronic acid. Multiple .gamma..delta.TCR isotypes may be used from any gamma delta TCR pairing from V.gamma.1-9 and V.delta.1-8. It will be understood by those of skill in the art that culture conditions, specifically the method of TCR activation, will define the isotype to be expanded. By way of example, .delta.2 T cells are activated and expanded by aminobisphosphonates and the like, whilst .delta.1 T cells may be preferentially expanded using NKG2D ligands such as MICA or MICB. Isolated PBMCs/CBMCs may be freshly isolated or cryopreserved prior to expansion in culture. In some embodiments expansion of CAR-gamma delta with regenerative properties is achieved by culture in bisphosphonates. A bisphosphonate is an analogue of pyrophosphoric acid and is a compound in which the 0 (oxygen atom) of the pyrophosphoric acid skeleton P—O—P is substituted with C (carbon atom) (P-C-P). It is generally used as a therapeutic drug for osteoporosis. The aminobisphosphonate refers to a compound having N (nitrogen atom) among the bisphosphonates. For example, the aminobisphosphonate used in the present invention is not particularly limited; examples thereof include pamidronic acid, its salt and/or their hydrate, alendronic acid, its salt and/or their hydrate, and zoledronic acid, its salt and/or their hydrate. The concentration of the aminobisphosphonates is preferably 1 to 30 .mu.M for pamidronic acid, its salt and/or their hydrate, 1 to 30 .mu.M for alendronic acid, its salt and/or their hydrate, and 0.1 to 10 .mu.M for zoledronic acid, its salt and/or their hydrate. Here, 5 .mu.M zoledronic acid is added as an example.

Suitably, the cytokine IL-2 may also be included at 50 IU/ml to 2000 IU/mL, more preferably 400 IU/mL to 1000 IU/mL. Suitably, the culture may also be supplemented with one or more cytokines such as IL-15, IL-18 or IL-21 at 50 IU/ml to 2000 IU/ml.

In some embodiments of the invention the gamma delta cells are reprogrammed by MSC to endow upon then therapeutic properties.

For the practice of the invention, MSC are used to reprogram immune cells in order to endow cardiac regenerative activity can be utilized as was previously performed in clinical trials with non-selected MSC. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

In some embodiments, dendritic cells are generated which possess tolerogenic activity and said dendritic cells are pulsed with cardiac antigens. Said cardiac antigens include the myosin heavy chain [27]. Said dendritic cells can be made tolerogenic by culture in cytokines such as IL-10 or they can be further made tolerogenic by culture with regenerative cells. In one embodiment said regenerative cells are mesenchymal stem cells. Generation of clinical grade dendritic cells is described in the following papers which are incorporated by reference [28-152].

Mesenchymal stem cells (MSCs) are adult stem cells with self-renewing abilities[153] and have been shown to differentiate into a wide range of tissues including mesoderm- and nonmesoderm-derived[153, 154], such as hepatocytes[155-160]. MSCs are capable of entering and maintaining satellite cell niches, particularly in hematopoiesis[161, 162], and are key in tissue repair and regeneration, aging, and regulating homeostasis[163-166]. In the case of heart failure, MSCs can aid in regeneration of cardiac tissue[167-173], and their interactions with the immune system[174-180] have potential as adjuvants during organ transplants[181], including cardiac transplantation[182].

MSCs were discovered in 1970 by Friedenstein et al[183] who demonstrated that bone marrow (BM) contained both hematopoietic stem cells (HSCs), which are non-plastic adherent, and a population of a more rare adherent cell. The adherent cells were able to form single cell colonies and were referred to as stromal cells. Those stromal cells, which are capable of self-renewal and expansion in culture are now referred to as mesenchymal stem cells (MSCs). Friedenstein was the first to show that MSCs could differentiate into mesoderm and to demonstrate their importance in controlling the hematopoietic niche [184].

In the 1980s, more research on MSCs found that they could differentiate into muscle, cartilage, bone and adipose-derived cells [185]. Caplan et al showed that MSCs are responsible for bone and cartilage regeneration induced by local cuing and genetic potential[186].

In the 1990s, Pittenger et al isolated MSCs from bone marrow and found that they retained their multilineage potential after expanding into selectively differentiated adipocytic, chondrocytic, or osteocytic lineages[154]. Likewise, Kopen et al showed that bone marrow MSCs differentiated into neural cells when exposed to the brain microenvironment[187]. In 1999, Petersen et al found that bone marrow-derived stem cells could be a source of hepatic oval cells in a rat model[188]. Specifically, they used male to female bone marrow transplant and subsequently induced blockade of hepatocyte proliferation by administration of a hepatotoxin followed by partial hepatectomy. As previously described, this procedure stimulates proliferation of LPC or “reserve cells” which generate new hepatocytes, such cells having previously identified as oval cells. Subsequent to the hepatectomy, Y chromosome, dipeptidyl peptidase IV enzyme, and L21-6 antigen were used to identify the newly generated oval cells, and their hepatocytic progeny to be of bone marrow origin.

The first decade of the 21′ century saw a surge of research on MSCs, leading to a greater understanding of their nature and of the cellular process behind regeneration [153, 165, 166]. In 2005, Teratani et al identified growth factors allowing hepatic fate-specification in mice and showed that embryonic stem cells could differentiate into functional hepatocytes [189]. A unique property of MSC is their apparent hypoimmunogenicity and immune modulatory activity [190], which is present in MSC derived from various sources [191]. This is believed to account for the ability to achieve therapeutic effects in an allogeneic manner. Allogeneic bone marrow derived MSC have been used by academic investigators with clinical benefit treatment of diseases such as graft versus host (GVHD) [192-197], osteogenesis imperfecta [198], Hurler syndrome, metachromatic leukodystrophy [199], and acceleration of hematopoietic stem cell engraftment [200-202]. The company Athersys has successfully completed Phase I safety studies using allogeneic bone marrow MSCs is now in efficacy finding clinical trials (Phase II and Phase III) for Multiple Sclerosis, Crohn's Disease, and Graft Versus Host Disease using allogeneic bone marrow derived MSC. Intravenous administration of allogeneic MSCs by Osiris was also reported to induce a statistically significant improvement in cardiac function in a double-blind study [203].

Currently there several MSC-based therapies that have received governmental approvals including Prochymal™ which was registered in Canada and New Zealand for treatment of graft versus host disease [204, 205]. Although in terms of clinical translation bone marrow MSC are the most advanced, several other sources of MSC are known which possess various properties that may be useful for specific conditions. Bone marrow is also a source for hematopoietic stem cells (HSCs), which have also been used for cardiac regeneration. Likewise, human placenta is an easily accessible source of abundant MSCs, which can be differentiated in vitro. Finally, MSCs with tissue regenerative abilities can also be isolated from adipose tissue and induced to hepatocytes in large numbers.

In some embodiments of the invention reprogrammed immune cells are administered with MSC for treatment of heart failure. The discussion below provides examples of the use of MSC in heart failure, which may be useful for one of skill in the art to combine MSC with reprogrammed immune cells

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIB ERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activities is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

In one embodiment, bone marrow MSC lots are generated, means of generating BM MSC are known in the literature and examples are incorporated by reference.

In one embodiment BM-MSC are generated as follows:

1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.
2. The total number of nucleated cells in the BM sample is counted by taking 10 .mu.L BM and diluting it 1/50-1/100 with 3% Acetic Acid with Methylene Blue (STEMCELL Catalog #07060). Cells are counted using a hemacytometer.
3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).
4. In three 50 mL conical tubes (BD Catalog #352070), 17 mL Ficoll-Paque.™. PLUS (Catalog #07907/07957) is pipetted into each tube. About 23 mL of the diluted BM from step 3 was carefully layered on top of the Ficoll-Paque.™. PLUS in each tube.
5. The tubes are centrifuged at room temperature (15-25.degree. C.) for 30 minutes at 300.times.g in a bench top centrifuge with the brake off.
6. The upper plasma layer is removed and discarded without disturbing the plasma:Ficoll-Paque.™. PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.
7. Cells were centrifuged at 300.times.g for 10 minutes at room temperature in a bench top centrifuge with the brake on. The supernatant is removed and the cell pellet resuspended in 1-2 mL cold Isolation Buffer.
8. Cells were diluted 1/50 in 3% Acetic Acid with Methylene Blue and the total number of nucleated cells counted using a hemacytometer.
9. Cells are diluted in Complete Human MesenCult.RTM.-Proliferation medium (STEMCELL catalog #05411) at a final concentration of 1.times.10.sup.6 cells/mL.
10. BM-derived cells were ready for expansion and CFU-F assays in the presence of GW2580, which can then be used for specific applications.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2'107 cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1'106 cells per ml in 175 cm2 tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1'106 per 175 cm2. Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm2 flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10'106 MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

Within the context of the invention, exosomes and microparticles may be used interchangeably. Exosomes from MSC may be generated from a mesenchymal stem cell conditioned medium (MSC-CM). Said exosomes are used in the context of the invention to reprogram immunocytes ex vivo or in vivo. Said particle may be isolated for example by being separated from non-associated components based on any property of the particle. For example, the particle may be isolated based on molecular weight, size, shape, composition or biological activity. The conditioned medium may be filtered or concentrated or both during, prior to or subsequent to separation. For example, it may be filtered through a membrane, for example one with a size or molecular weight cut-off. It may be subject to tangential force filtration or ultrafiltration. Filtration of conditioned media is described in the following and incorporated by reference [206]. For example, filtration with a membrane of a suitable molecular weight or size cutoff. The conditioned medium, optionally filtered or concentrated or both, may be subject to further separation means, such as column chromatography. For example, high performance liquid chromatography (HPLC) with various columns may be used. The columns may be size exclusion columns or binding columns. One or more properties or biological activities of the particle may be used to track its activity during fractionation of the mesenchymal stem cell conditioned medium (MSC-CM). As an example, light scattering, refractive index, dynamic light scattering or UV-visible detectors may be used to follow the particles. For example, a therapeutic activity such as antirheumatic activity may be used to track the activity during fractionation. In one embodiment antirheumatic activity is assessed by ability to inhibit TNF-alpha production from stimulated monocytes or monocytic lineage cell such as macrophages or dendritic cells.

In one aspect of the invention MSC are cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrane. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymal stem cell particles such as exosomes.

MSC can be prepared from a variety of tissues, such as bone marrow cells [207-213], umbilical cord tissue [214-216], peripheral blood [217-219], amniotic membrane [220], amniotic fluid, mobilized peripheral blood [221], adipose tissue [222, 223], endometrium and other tissues. When tissue sources of MSC are used said tissue isolates from which the Reprogrammed immune cells are isolated comprise a mixed populations of cells. Reprogrammed immune cells constitute a very small percentage in these initial populations. They must be purified away from the other cells before they can be expanded in culture sufficiently to obtain enough cells for therapeutic applications.

For expansion of gamma delta T cells, antigen provision via aminobisphosphonate may be substituted with synthetic antigens such as isopentenyl pyrophosphate (IPP), phosphostim/bromohydrin pyrophosphate (BrHPP), (E)-4-Hydroxy-3-methyl-but-2-enyl pyrophosphate (HMBPP) or DMAPP. Antigenic stimulation may also be provided by co-culturing with irradiated and/or artificial antigen presenting cells (aAPC). The addition of such components provides a culturing environment which allows for positive selection of gamma delta T cells typically at 70%-100% by number of total cells in the culture sample. The expansion of .gamma..delta.T cells may be also stimulated with one or more antibodies against CD3, gamma delta TCR, CD28 and CD137. These can be soluble, plate bound or conjugated to appropriate beads, such as dynabeads or MACSibeads. .gamma..delta.T cells may be propagated and expanded using any of the methodologies previously disclosed. CAR-expressing cells can be selectively expanded with the use of a recombinant protein recognised by the CAR e.g. recombinant CD19-Fc chimera or recombinant CD19 in the case of a CD19-targeted CAR. This can be plate bound, soluble or on appropriate beads, such as dynabeads or MACSibeads. .alpha..delta.T cells of any isotype may be selectively expanded for a time-frame of at least 7 days, more preferably 14 days. Suitably, the period of culturing may be performed for about 9 days or greater to achieve high numbers of substantially purified CAR-expressing gamma delta T cell populations. .gamma..delta.T cells may be expanded using TCR antigens or NKG2D ligands such as MICA or MICB. Isolated PBMCs may be freshly isolated or cryopreserved prior to expansion in culture. Expanded .gamma..delta.T cells may be cryopreserved and resuscitated at a later time point for further expansion in culture.

The method of genetically modifying a .gamma..delta.T cell to incorporate the nucleic acid encoding a CAR design may include any technique known to those skilled in the art. Suitable methodologies include, but are not restricted to, viral transduction with lentiviruses/retroviruses/adenoviruses, cellular transfection by electroporation, lipid-based transfection reagents, nanoparticles, calcium chloride based transfection methods or bacterially-derived transposons.

For the practice of the invention, a CAR is generally considered to redirect the T cell specificity to the cell surface target and overcomes issues relating to T cell tolerance, but instead of inducing the T cell to cause injury, the regenerative-CAR is utilized to stimulate tissue repair. As will be appreciated, the cell surface target typically can be selected to ensure targeting of the gamma delta T cell towards cells of interest such as injured cardiac, neural or other tissue type of cell. It is known in the art that CAR targeting systems where the chimeric antigen receptor fuse signal 1 and signal 2 components into a single construct, they can provide highly potent and sensitive target-dependent effector responses. However, such CAR targeting systems are not tuneable to the level of cell surface target expressed on a target cell. It is considered that V.gamma.9V.delta.2 TCR-mediated recognition provides a further CAR independent targeting strategy that allows the CAR response to be “tuned” such that a stimulating signal from the CAR will translate to a functional response only in the context of V.gamma.9V.delta.2 TCR stimulation. This allows, for example a broad range of degenerated targets target cells (HMBPP/IPP stress related pathway targets) to be targeted with an additional cell surface target by a CAR-modified gamma delta T cell. These CAR modified gamma delta T cells of the invention provide a tunable response, wherein for a given tumour-associated antigen, pyrophosphate/phosphonate (drug) dose there is generated a suboptimal signal 1 strength response, with an ability to synergize with signal 2 from a specific “co-stimulatory CAR”.[0043] In embodiments a gamma delta T cell can further comprise an inhibitory chimeric antigen receptor (ICAR), wherein the ICAR minimises activation in off-target cells e.g. non tumour cells wherein the cell surface target is a tumour-associated, but not tumour-specific antigen. To minimise for example, such “on target, off tumour toxicity” the presence of a second antigen on an off target cell which can be bound by the inhibitory CAR will cause the signal provided by any binding of the CAR to the cell surface target to be inhibited. In some embodiments, the gamma delta cell can comprise a further CAR capable of binding to a different antigen present on a target cell or to soluble signalling proteins present in.

In embodiments the gamma delta T cell with at least a first chimeric antigen receptor and optionally at least a first inhibitory chimeric antigen receptor, is a V.gamma.9V.delta.2 T cell. Provision of chimeric antigen receptor to a gamma delta T cell can be by means known in the art to provide chimeric antigen receptors to T cells. Accordingly to a fourth aspect of the invention, there is provided a process to provide a CAR modified .gamma..delta.T cell wherein the nucleic acid of the third aspect is incorporated/transduced into a .gamma..delta.T cell to genetically modify the .gamma..delta.T cell. Suitably, the process can utilise a lentiviral CAR construct. Suitably, the process simultaneously transduces a CAR construct into a cell, and selectively expands CAR-transduced .gamma..delta.T cells. In some embodiments, it is considered embodiments of the process will enable a ‘TCR-tuneable’ or ‘co-stimulatory’ CAR to be provided.

As discussed above, in such embodiments, .gamma..delta.T cells expressing a co-stimulatory CAR will be activated only in the presence of phosphoantigens (present on the cell surface during infection or cancer) but not by healthy cells. It is considered this will circumvent the “on-target” but “off-tumour” toxicity observed in conventional CAR-T therapies. In such embodiments, it is considered the activity of the co-stimulatory CAR can be tuned by concomitant TCR signalling through the .gamma..delta.T cell receptor.

In embodiments of the third aspect of the invention, the nucleic acid sequence encoding a CAR may include a leader sequence which will direct the protein to the cell membrane (such as the GMCSF-R secretory signal or CD8), an antigen binding domain, a hinge and transmembrane domain, one or more co-stimulatory signalling regions and the presence or absence of a CD3 zeta signalling domain. As discussed herein, the absence of a CD3 zeta signalling domain may be provided by an inactive or non-functional CD3 zeta domain. In embodiments of the nucleic acid including the CD3 zeta domain, the CAR is considered ‘classical’ or ‘non-tuneable’. In embodiments in which the CD3 zeta domain has been omitted, the CAR is a ‘co-stimulatory’ or ‘TCR-tuneable’ CAR.

### Example 1: Generation of Gamma Delta T Cells

Peripheral blood mononuclear cells (PBMC) were isolated from purchased whole blood leucocyte cones via density gradient centrifugation using Lymphoprep (Stemcell) according to manufacturer's instruction. PBMC cryopreserved in 90% FBS 10% DMSO.

To generate T cell cultures, cells were thawed and re-suspended in complete T cell culture media for further processing. Complete T cell culture media consisted of xeno- and serum-free CTS-OpTmizer (Thermo Fisher) with 10% synthetic serum replacement (Thermo Fisher) and GlutaMAX (Thermo Fisher), PBMC were thawed and rested at 10×106 cells/mL in complete pre-warmed media overnight before further processing to avoid over-stressing the lymphocytes and to enhance depletion quality. PB MC at 2-4×106 cells/mL density were then either stimulated in standard cell culture plates right away or first depleted of αβT cells using the TCRα/β Product Line (Miltenyi Biotec) according to manufacturer's instructions concurrently with depletion of CD56-positive cells using CD56 MicroBeads (Miltenyi Biotec) according to manufacturer's instructions.

Briefly, cells were first labelled with anti-TCRα/β-biotin, then a mix of anti-biotin microbeads and anti-CD56 beads, and then depleted using MACS Cell Separation LD Columns (Miltenyi Biotec).

If cultured in G-Rex vessels (Wilson Wolf), depleted PBMC were initiated at 2-4×106 cells/cm2. Thus-prepared PBMC were stimulated with either 1 μg/mL OKT-3 (Miltenyi Biotec Cat #130-093-387, RRID: AB_1036144) or 1 μg/mL PHA (Merck) and various cytokine combinations: (i) 100 IU/mL IL-2 aldesleukin (Proleukin; Novartis), (ii) 70 ng/mL IL-15 (Peprotech), (iii) 20 ng/mL rhIL-7 (Peprotech), or the (iv) ‘DOT protocol’ cytokine cocktail, which consisted of a first culture in 100 ng/mL rIL-4, 70 ng/mL rIFN-γ, 7 ng/mL rIL-21 and 15 ng/mL rIL-1β followed by a second culture in 70 ng/mL rIL-15 and 30 ng/mL IFN-γ (all from Peprotech).

Briefly, depleted PBMC were stimulated for a first cytokine culture with 70 ng/mL OKT-3, and then a second cytokine culture with 1 μg/mL OKT-3. Live cells before and during expansion were counted using Trypan Blue exclusion, an automatic cell counter (Invitrogen) and flow cytometry-based Precision Count Beads (Biolegend).

### Example 2: Regenerative Properties of Untransfected Gamma Delta T Cells

Gamma Delta T cells were obtained as described in Example 1 and cultured together with human umbilical vein endothelial cells (HUVEC) at the indicated ratios. HUVEC cells were purchased from ATCC and grown in Optimem Media with 10% fetal calf serum. Cells were cultured alone or contacted for the indicated times and then proliferation was assessed by tritiated thymidine incorporation for 24 hours. As seen in the figure below, Gamma Delta T cells stimulated a potent proliferative response from HUVEC cells, which is the established model of angiogenesis and regeneration.

### REFERENCES

1. Jorgensen, N., et al., Peripheral blood immune markers in breast cancer. Differences in regulatory T cell abundance are related to clinical parameters. Clin Immunol, 2021. 232: p. 108847.
2. Ribot, J. C., N. Lopes, and B. Silva-Santos, gammadelta T cells in tissue physiology and surveillance. Nat Rev Immunol, 2021. 21 (4): p. 221-232.
3. Pamplona, A. and B. Silva-Santos, gammadelta T cells in malaria: a double-edged sword. FEBS J, 2021. 288 (4): p. 1118-1129.
4. Yang, X., et al., Tofacitinib restores the balance of gammadeltaTreg/gammadeltaT17 cells in rheumatoid arthritis by inhibiting the NLRP3 inflammasome. Theranostics, 2021. 11 (3): p. 1446-1457.
5. Du, B., et al., The prostaglandin E2 increases the production of IL-17 and the expression of costimulatory molecules on gammadelta T cells in rheumatoid arthritis. Scand J Immunol, 2020. 91 (5): p. e12872.
6. Schwaneck, E. C., et al., T cells, natural killer cells, and gammadeltaT cells in a large patient cohort with rheumatoid arthritis: influence of age and anti-rheumatic therapy. Scand J Rheumatol, 2020. 49 (1): p. 8-12.
7. Collins, C., et al., Detection of Cell Surface Ligands for Human Synovial gammadelta T Cells. J Immunol, 2019. 203 (9): p. 2369-2376.
8. Bouchareychas, L., et al., gammadeltaTCR regulates production of interleukin-27 by neutrophils and attenuates inflammatory arthritis. Sci Rep, 2018. 8 (1): p. 7590.
9. Rossini, M., et al., Rheumatoid arthritis, gammadelta T cells and bisphosphonates. Ann Rheum Dis, 2018. 77 (9): p. e57.
10. Kessel, C., et al., Proinflammatory Cytokine Environments Can Drive Interleukin-17 Overexpression by gamma/delta T Cells in Systemic Juvenile Idiopathic Arthritis. Arthritis Rheumatol, 2017. 69 (7): p. 1480-1494.
11. Wenink, M. H., et al., Review: Innate Lymphoid Cells: Sparking Inflammatory Rheumatic Disease? Arthritis Rheumatol, 2017. 69 (5): p. 885-897.
12. Andersson, A., et al., IL-17-producing gammadeltaT cells are regulated by estrogen during development of experimental arthritis. Clin Immunol, 2015. 161 (2): p. 324-32.
13. Gaur, P., R. Misra, and A. Aggarwal, Natural killer cell and gamma delta T cell alterations in enthesitis related arthritis category of juvenile idiopathic arthritis. Clin Immunol, 2015. 161 (2): p. 163-9.
14. Akitsu, A., et al., IL-1 receptor antagonist-deficient mice develop autoimmune arthritis due to intrinsic activation of IL-17-producing CCR2(+)Vgamma6(+)gammadelta T cells. Nat Commun, 2015. 6: p. 7464.
15. Divan, A., et al., gammadelta T Cells and dendritic cells in refractory Lyme arthritis. J Leukoc Biol, 2015. 97 (4): p. 653-63.
16. Laurent, A. J., et al., Synergistic effects of ethanol and isopentenyl pyrophosphate on expansion of gammadelta T cells in synovial fluid from patients with arthritis. PLoS One, 2014. 9 (8): p. e103683.
17. Su, D., et al., (99) Tc-methylene diphosphonate improves rheumatoid arthritis disease activity by increasing the frequency of peripheral gammadelta T cells and CD4(+) CD25(+) Foxp3(+) Tregs. Int J Rheum Dis, 2016. 19 (6): p. 586-93.
18. Walscheid, K., et al., Increased Circulating Proinflammatory T Lymphocytes in Children with Different Forms of Anterior Uveitis: Results from a Pilot Study. Ocul Immunol Inflamm, 2019. 27 (5): p. 788-797.
19. Kim, A., et al., The Role of Th-17 Cells and gammadelta T-Cells in Modulating the Systemic Inflammatory Response to Severe Burn Injury. Int J Mol Sci, 2017. 18 (4).
20. Hasan, M. S., et al., Gamma Delta (gammadelta) T Cells and Their Involvement in Behcet's Disease. J Immunol Res, 2015. 2015: p. 705831.
21. Lewis, E. L., et al., IFNgamma-Producing gamma/delta T Cells Accumulate in the Fetal Brain Following Intrauterine Inflammation. Front Immunol, 2021. 12: p. 741518.
22. Anh, B. V., et al., Vgamma9gammadelta T Cell Induction by Human Umbilical Cord Blood Monocytes-Derived, Interferon-alpha-Stimulated Dendritic Cells. Cancer Control, 2020. 27 (1): p. 1073274820974025.
23. Kang, S., et al., Tissue Resident Memory gammadelta T Cells in Murine Uterus Expressed High Levels of IL-17 Promoting the Invasion of Trophocytes. Front Immunol, 2020. 11: p. 588227.
24. Sagar, et al., Deciphering the regulatory landscape of fetal and adult gammadelta T-cell development at single-cell resolution. EMBO J, 2020. 39 (13): p. e104159.
25. Terzieva, A., et al., Early Pregnancy Human Decidua is Enriched with Activated, Fully Differentiated and Pro-Inflammatory Gamma/Delta T Cells with Diverse TCR Repertoires. Int J Mol Sci, 2019. 20 (3).
26. Engelmann, I., et al., Activation status of cord blood gamma delta T cells reflects in utero exposure to Plasmodium falciparum antigen. J Infect Dis, 2005. 191 (10): p. 1612-22.
27. DeBerge, M., et al., Monocytes prime autoreactive T cells after myocardial infarction. Am J Physiol Heart Circ Physiol, 2020. 318 (1): p. H116-H123.
28. Nestle, F. O., et al., Vaccination of melanoma patients with peptide- or tumor lysate-pulsed dendritic cells. Nat Med, 1998. 4 (3): p. 328-32.
29. Chakraborty, N. G., et al., Immunization with a tumor-cell-lysate-loaded autologous-antigen-presenting-cell-based vaccine in melanoma. Cancer Immunol Immunother, 1998. 47 (1): p. 58-64.
30. Wang, F., et al., Phase I trial of a MART-1 peptide vaccine with incomplete Freund's adjuvant for resected high-risk melanoma. Clin Cancer Res, 1999. 5 (10): p. 2756-65.
31. Thurner, B., et al., Vaccination with mage-3A1 peptide pulsed mature, monocyte-derived dendritic cells expands specific cytotoxic T cells and induces regression of some metastases in advanced stage IV melanoma. J Exp Med, 1999. 190 (11): p. 1669-78.
32. Thomas, R., et al., Immature human monocyte-derived dendritic cells migrate rapidly to draining lymph nodes after intradermal injection for melanoma immunotherapy. Melanoma Res, 1999. 9 (5): p. 474-81.
33. Mackensen, A., et al., Phase I study in melanoma patients of a vaccine with peptide-pulsed dendritic cells generated in vitro from CD34(+) hematopoietic progenitor cells. Int J Cancer, 2000. 86 (3): p. 385-92.
34. Panelli, M. C., et al., Phase 1 study in patients with metastatic melanoma of immunization with dendritic cells presenting epitopes derived from the melanoma-associated antigens MART-1 and gp100. J Immunother, 2000. 23 (4): p. 487-98.
35. Schuler-Thurner, B., et al., Mage-3 and influenza-matrix peptide-specific cytotoxic T cells are inducible in terminal stage HLA-A2.1+ melanoma patients by mature monocyte-derived dendritic cells. J Immunol, 2000. 165 (6): p. 3492-6.
36. Lau, R., et al., Phase I trial of intravenous peptide-pulsed dendritic cells in patients with metastatic melanoma. J Immunother, 2001. 24 (1): p. 66-78.
37. Banchereau, J., et al., Immune and clinical responses in patients with metastatic melanoma to CD34(+) progenitor-derived dendritic cell vaccine. Cancer Res, 2001. 61 (17): p. 6451-8.
38. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195 (10): p. 1279-88.
39. Palucka, A. K., et al., Single injection of CD34+ progenitor-derived dendritic cell vaccine can lead to induction of T-cell immunity in patients with stage IV melanoma. J Immunother, 2003. 26 (5): p. 432-9.
40. Bedrosian, I., et al., Intranodal administration of peptide pulsed mature dendritic cell vaccines results in superior CD8+T-cell function in melanoma patients. J Clin Oncol, 2003. 21 (20): p. 3826-35.
41. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21 (21): p. 4016-26.
42. Hersey, P., et al., Phase I/II study of treatment with dendritic cell vaccines in patients with disseminated melanoma. Cancer Immunol Immunother, 2004. 53 (2): p. 125-34.
43. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53 (7): p. 651-8.
44. Palucka, A. K., et al., Spontaneous proliferation and type 2 cytokine secretion by CD4+T cells in patients with metastatic melanoma vaccinated with antigen-pulsed dendritic cells. J Clin Immunol, 2005. 25 (3): p. 288-95.
45. Banchereau, J., et al., Immune and clinical outcomes in patients with stage IV melanoma vaccinated with peptide-pulsed dendritic cells derived from CD34+ progenitors and activated with type I interferon. J Immunother, 2005. 28 (5): p. 505-16.
46. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55 (4): p. 469-74.
47. Salcedo, M., et al., Vaccination of melanoma patients using dendritic cells loaded with an allogeneic tumor cell lysate. Cancer Immunol Immunother, 2006. 55 (7): p. 819-29.
48. Linette, G. P., et al., Immunization using autologous dendritic cells pulsed with the melanoma-associated antigen gp100-derived G280-9V peptide elicits CD8+ immunity. Clin Cancer Res, 2005. 11 (21): p. 7692-9.
49. Escobar, A., et al., Dendritic cell immunizations alone or combined with low doses of interleukin-2 induce specific immune responses in melanoma patients. Clin Exp Immunol, 2005. 142 (3): p. 555-68.
50. Tuettenberg, A., et al., Induction of strong and persistent MelanA/MART-1-specific immune responses by adjuvant dendritic cell-based vaccination of stage II melanoma patients. Int J Cancer, 2006. 118 (10): p. 2617-27.
51. Schadendorf, D., et al., Dacarbazine (DTIC) versus vaccination with autologous peptide-pulsed dendritic cells (DC) in first-line treatment of patients with metastatic melanoma: a randomized phase III trial of the DC study group of the DeCOG. Ann Oncol, 2006. 17 (4): p. 563-70.
52. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66 (9): p. 4943-51.
53. Nakai, N., et al., Vaccination of Japanese patients with advanced melanoma with peptide, tumor lysate or both peptide and tumor lysate pulsed mature, monocyte-derived dendritic cells. J Dermatol, 2006. 33 (7): p. 462-72.
54. Palucka, A. K., et al., Dendritic cells loaded with killed allogeneic melanoma cells can induce objective clinical responses and MART-1 specific CD8+T-cell immunity. J Immunother, 2006. 29 (5): p. 545-57.
55. Lesimple, T., et al., Immunologic and clinical effects of injecting mature peptide-loaded dendritic cells by intralymphatic and intranodal routes in metastatic melanoma patients. Clin Cancer Res, 2006. 12 (24): p. 7380-8.
56. Guo, J., et al., Intratumoral injection of dendritic cells in combination with local hyperthermia induces systemic antitumor effect in patients with advanced melanoma. Int J Cancer, 2007. 120 (11): p. 2418-25.
57. O'Rourke, M. G., et al., Dendritic cell immunotherapy for stage IV melanoma. Melanoma Res, 2007. 17 (5): p. 316-22.
58. Bercovici, N., et al., Analysis and characterization of antitumor T-cell response after administration of dendritic cells loaded with allogeneic tumor lysate to metastatic melanoma patients. J Immunother, 2008. 31 (1): p. 101-12.
59. Hersey, P., et al., Phase I/II study of treatment with matured dendritic cells with or without low dose IL-2 in patients with disseminated melanoma. Cancer Immunol Immunother, 2008. 57 (7): p. 1039-51.
60. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.
61. Carrasco, J., et al., Vaccination of a melanoma patient with mature dendritic cells pulsed with MAGE-3 peptides triggers the activity of nonvaccine anti-tumor cells. J Immunol, 2008. 180 (5): p. 3585-93.
62. Redman, B. G., et al., Phase Ib trial assessing autologous, tumor-pulsed dendritic cells as a vaccine administered with or without IL-2 in patients with metastatic melanoma. J Immunother, 2008. 31 (6): p. 591-8.
63. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26 (19): p. 3235-41.
64. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58 (1): p. 1-14.
65. Nakai, N., et al., Immunohistological analysis of peptide-induced delayed-type hypersensitivity in advanced melanoma patients treated with melanoma antigen pulsed mature monocyte-derived dendritic cell vaccination. J Dermatol Sci, 2009. 53 (1): p. 40-7.
66. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24 (3): p. 311-9.
67. Chang, J. W., et al., Immunotherapy with dendritic cells pulsed by autologous dactinomycin-induced melanoma apoptotic bodies for patients with malignant melanoma. Melanoma Res, 2009. 19 (5): p. 309-15.
68. Trepiakas, R., et al., Vaccination with autologous dendritic cells pulsed with multiple tumor antigens for treatment of patients with malignant melanoma: results from a phase I/II trial. Cytotherapy, 2010. 12 (6): p. 721-34.
69. Jacobs, J. F., et al., Dendritic cell vaccination in combination with anti-CD25 monoclonal antibody treatment: a phase I/II study in metastatic melanoma patients. Clin Cancer Res, 2010. 16 (20): p. 5067-78.
70. Ribas, A., et al., Multicenter phase II study of matured dendritic cells pulsed with melanoma cell line lysates in patients with advanced melanoma. J Transl Med, 2010. 8: p. 89.
71. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.
72. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60 (1): p. 123-31.
73. Lesterhuis, W. J., et al., Wild-type and modified gp100 peptide-pulsed dendritic cell vaccination of advanced melanoma patients can lead to long-term clinical responses independent of the peptide used. Cancer Immunol Immunother, 2011. 60 (2): p. 249-60.
74. Bjoern, J., et al., Changes in peripheral blood level of regulatory T cells in patients with malignant melanoma during treatment with dendritic cell vaccination and low-dose IL-2. Scand J Immunol, 2011. 73 (3): p. 222-33.
75. Steele, J. C., et al., Phase I/II trial of a dendritic cell vaccine transfected with DNA encoding melan A and gp100 for patients with metastatic melanoma. Gene Ther, 2011. 18 (6): p. 584-93.
76. Kim, D. S., et al., Immunotherapy of malignant melanoma with tumor lysate-pulsed autologous monocyte-derived dendritic cells. Yonsei Med J, 2011. 52 (6): p. 990-8.
77. Ellebaek, E., et al., Metastatic melanoma patients treated with dendritic cell vaccination, Interleukin-2 and metronomic cyclophosphamide: results from a phase II trial. Cancer Immunol Immunother, 2012. 61 (10): p. 1791-804.
78. Dillman, R. O., et al., Tumor stem cell antigens as consolidative active specific immunotherapy: a randomized phase II trial of dendritic cells versus tumor cells in patients with metastatic melanoma. J Immunother, 2012. 35 (8): p. 641-9.
79. Dannull, J., et al., Melanoma immunotherapy using mature DCs expressing the constitutive proteasome. J Clin Invest, 2013. 123 (7): p. 3135-45.
80. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82 (2): p. 924-32.
81. Stift, A., et al., Dendritic cell vaccination in medullary thyroid carcinoma. Clin Cancer Res, 2004. 10 (9): p. 2944-53.
82. Kuwabara, K., et al., Results of a phase I clinical study using dendritic cell vaccinations for thyroid cancer. Thyroid, 2007. 17 (1): p. 53-8.
83. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21 (6): p. 1585-92.
84. Yu, J. S., et al., Vaccination of malignant glioma patients with peptide-pulsed dendritic cells elicits systemic cytotoxicity and intracranial T-cell infiltration. Cancer Res, 2001. 61 (3): p. 842-7.
85. Yamanaka, R., et al., Vaccination of recurrent glioma patients with tumour lysate pulsed dendritic cells elicits immune responses: results of a clinical phase I/II trial. Br J Cancer, 2003. 89 (7): p. 1172-9.
86. Yu, J. S., et al., Vaccination with tumor lysate-pulsed dendritic cells elicits antigen-specific, cytotoxic T-cells in patients with malignant glioma. Cancer Res, 2004. 64 (14): p. 4973-9.
87. Yamanaka, R., et al., Tumor lysate and IL-18 loaded dendritic cells elicits Th1 response, tumor-specific CD8+ cytotoxic T cells in patients with malignant glioma. J Neurooncol, 2005. 72 (2): p. 107-13.
88. Yamanaka, R., et al., Clinical evaluation of dendritic cell vaccination for patients with recurrent glioma: results of a clinical phase I/II trial. Clin Cancer Res, 2005. 11 (11): p. 4160-7.
89. Liau, L. M., et al., Dendritic cell vaccination in glioblastoma patients induces systemic and intracranial T-cell responses modulated by the local central nervous system tumor microenvironment. Clin Cancer Res, 2005. 11 (15): p. 5515-25.
90. Walker, D. G., et al., Results of a phase I dendritic cell vaccine trial for malignant astrocytoma: potential interaction with adjuvant chemotherapy. J Clin Neurosci, 2008. 15 (2): p. 114-21.
91. Leplina, O. Y., et al., Use of interferon-alpha-induced dendritic cells in the therapy of patients with malignant brain gliomas. Bull Exp Biol Med, 2007. 143 (4): p. 528-34.
92. De Vleeschouwer, S., et al., Postoperative adjuvant dendritic cell-based immunotherapy in patients with relapsed glioblastoma multiforme. Clin Cancer Res, 2008. 14 (10): p. 3098-104.
93. Ardon, H., et al., Adjuvant dendritic cell-based tumour vaccination for children with malignant brain tumours. Pediatr Blood Cancer, 2010. 54 (4): p. 519-25.
94. Prins, R. M., et al., Gene expression profile correlates with T-cell infiltration and relative survival in glioblastoma patients vaccinated with dendritic cell immunotherapy. Clin Cancer Res, 2011. 17 (6): p. 1603-15.
95. Okada, H., et al., Induction of CD8+T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol, 2011. 29 (3): p. 330-6.
96. Fadul, C. E., et al., Immune response in patients with newly diagnosed glioblastoma multiforme treated with intranodal autologous tumor lysate-dendritic cell vaccination after radiation chemotherapy. J Immunother, 2011. 34 (4): p. 382-9.
97. Chang, C. N., et al., A phase I/II clinical trial investigating the adverse and therapeutic effects of a postoperative autologous dendritic cell tumor vaccine in patients with malignant glioma. J Clin Neurosci, 2011. 18 (8): p. 1048-54.
98. Cho, D. Y., et al., Adjuvant immunotherapy with whole-cell lysate dendritic cells vaccine for glioblastoma multiforme: a phase II clinical trial. World Neurosurg, 2012. 77 (5-6): p. 736-44.
99. Iwami, K., et al., Peptide-pulsed dendritic cell vaccination targeting interleukin-13 receptor alpha2 chain in recurrent malignant glioma patients with HLA-A*24/A*02 allele. Cytotherapy, 2012. 14 (6): p. 733-42.
100. Fong, B., et al., Monitoring of regulatory T cell frequencies and expression of CTLA-4 on T cells, before and after DC vaccination, can predict survival in GBM patients. PLoS One, 2012. 7 (4): p. e32614.
101. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61 (11): p. 2105-12.
102. Phuphanich, S., et al., Phase I trial of a multi-epitope-pulsed dendritic cell vaccine for patients with newly diagnosed glioblastoma. Cancer Immunol Immunother, 2013. 62 (1): p. 125-35.
103. Akiyama, Y., et al., alpha-type-1 polarized dendritic cell-based vaccination in recurrent high-grade glioma: a phase I clinical trial. BMC Cancer, 2012. 12: p. 623.
104. Prins, R. M., et al., Comparison of glioma-associated antigen peptide-loaded versus autologous tumor lysate-loaded dendritic cell vaccination in malignant glioma patients. J Immunother, 2013. 36 (2): p. 152-7.
105. Shah, A. H., et al., Dendritic cell vaccine for recurrent high-grade gliomas in pediatric and adult subjects: clinical trial protocol. Neurosurgery, 2013. 73 (5): p. 863-7.
106. Reichardt, V. L., et al., Idiotype vaccination using dendritic cells after autologous peripheral blood stem cell transplantation for multiple myeloma—a feasibility study. Blood, 1999. 93 (7): p. 2411-9.
107. Lim, S. H. and R. Bailey-Wood, Idiotypic protein-pulsed dendritic cell vaccination in multiple myeloma. Int J Cancer, 1999. 83 (2): p. 215-22.
108. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121 (2): p. 240-50.
109. Reichardt, V. L., et al., Idiotype vaccination of multiple myeloma patients using monocyte-derived dendritic cells. Haematologica, 2003. 88 (10): p. 1139-49.
110. Guardino, A. E., et al., Production of myeloid dendritic cells (DC) pulsed with tumor-specific idiotype protein for vaccination of patients with multiple myeloma. Cytotherapy, 2006. 8 (3): p. 277-89.
111. Lacy, M. Q., et al., Idiotype-pulsed antigen presenting cells following autologous transplantation for multiple myeloma may be associated with prolonged survival. Am J Hematol, 2009. 84 (12): p. 799-802.
112. Yi, Q., et al., Optimizing dendritic cell-based immunotherapy in multiple myeloma: intranodal injections of idiotype pulsed CD40 ligand-matured vaccines led to induction of type-1 and cytotoxic T-cell immune responses in patients. Br J Haematol, 2010. 150 (5): p. 554-64.
113. Rollig, C., et al., Induction of cellular immune responses in patients with stage-I multiple myeloma after vaccination with autologous idiotype-pulsed dendritic cells. J Immunother, 2011. 34 (1): p. 100-6.
114. Zahradova, L., et al., Efficacy and safety of Id-protein-loaded dendritic cell vaccine in patients with multiple myeloma—phase II study results. Neoplasma, 2012. 59 (4): p. 440-9.
115. Timmerman, J. M., et al., Idiotype pulsed dendritic cell vaccination for B-cell lymphoma: clinical and immune responses in 35 patients. Blood, 2002. 99 (5): p. 1517-26.
116. Maier, T., et al., Vaccination of patients with cutaneous T-cell lymphoma using intranodal injection of autologous tumor-lysate-pulsed dendritic cells. Blood, 2003. 102 (7): p. 2338-44.
117. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113 (1): p. 18-27.
118. Hus, I., et al., Allogeneic dendritic cells pulsed with tumor lysates or apoptotic bodies as immunotherapy for patients with early-stage B-cell chronic lymphocytic leukemia. Leukemia, 2005. 19 (9): p. 1621-7.
119. Li, L., et al., Immunotherapy for patients with acute myeloid leukemia using autologous dendritic cells generated from leukemic blasts. Int J Oncol, 2006. 28 (4): p. 855-61.
120. Roddie, H., et al., Phase I/II study of vaccination with dendritic-like leukaemia cells for the immunotherapy of acute myeloid leukaemia. Br J Haematol, 2006. 133 (2): p. 152-7.
121. Litzow, M. R., et al., Testing the safety of clinical-grade mature autologous myeloid DC in a phase I clinical immunotherapy trial of CML. Cytotherapy, 2006. 8 (3): p. 290-8.
122. Westermann, J., et al., Vaccination with autologous non-irradiated dendritic cells in patients with bcr/abl+ chronic myeloid leukaemia. Br J Haematol, 2007. 137 (4): p. 297-306.
123. Hus, I., et al., Vaccination of B-CLL patients with autologous dendritic cells can change the frequency of leukemia antigen-specific CD8+ T cells as well as CD4+CD25+FoxP3+ regulatory T cells toward an antileukemia response. Leukemia, 2008. 22 (5): p. 1007-17.
124. Palma, M., et al., Development of a dendritic cell-based vaccine for chronic lymphocytic leukemia. Cancer Immunol Immunother, 2008. 57 (11): p. 1705-10.
125. Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107 (31): p. 13824-9.
126. Iwashita, Y., et al., A phase I study of autologous dendritic cell-based immunotherapy for patients with unresectable primary liver cancer. Cancer Immunol Immunother, 2003. 52 (3): p. 155-61.
127. Lee, W. C., et al., Vaccination of advanced hepatocellular carcinoma patients with tumor lysate-pulsed dendritic cells: a clinical trial. J Immunother, 2005. 28 (5): p. 496-504.
128. Butterfield, L. H., et al., A phase I/II trial testing immunization of hepatocellular carcinoma patients with dendritic cells pulsed with four alpha-fetoprotein peptides. Clin Cancer Res, 2006. 12 (9): p. 2817-25.
129. Palmer, D. H., et al., A phase II study of adoptive immunotherapy using dendritic cells pulsed with tumor lysate in patients with hepatocellular carcinoma. Hepatology, 2009. 49 (1): p. 124-32.
130. El Ansary, M., et al., Immunotherapy by autologous dendritic cell vaccine in patients with advanced HCC. J Cancer Res Clin Oncol, 2013. 139 (1): p. 39-48.
131. Tada, F., et al., Phase I/II study of immunotherapy using tumor antigen-pulsed dendritic cells in patients with hepatocellular carcinoma. Int J Oncol, 2012. 41 (5): p. 1601-9.
132. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24 (4): p. 909-17.
133. Hirschowitz, E. A., et al., Autologous dendritic cell vaccines for non-small-cell lung cancer. J Clin Oncol, 2004. 22 (14): p. 2808-15.
134. Chang, G. C., et al., A pilot clinical trial of vaccination with dendritic cells pulsed with autologous tumor cells derived from malignant pleural effusion in patients with late-stage lung carcinoma. Cancer, 2005. 103 (4): p. 763-71.
135. Yannelli, J. R., et al., The large scale generation of dendritic cells for the immunization of patients with non-small cell lung cancer (NSCLC). Lung Cancer, 2005. 47 (3): p. 337-50.
136. Ishikawa, A., et al., A phase I study of alpha-galactosylceramide (KRN7000)-pulsed dendritic cells in patients with advanced and recurrent non-small cell lung cancer. Clin Cancer Res, 2005. 11 (5): p. 1910-7.
137. Antonia, S. J., et al., Combination of p53 cancer vaccine with chemotherapy in patients with extensive stage small cell lung cancer. Clin Cancer Res, 2006. 12 (3 Pt 1): p. 878-87.
138. Perrot, I., et al., Dendritic cells infiltrating human non-small cell lung cancer are blocked at immature stage. J Immunol, 2007. 178 (5): p. 2763-9.
139. Hirschowitz, E. A., et al., Immunization of NSCLC patients with antigen-pulsed immature autologous dendritic cells. Lung Cancer, 2007. 57 (3): p. 365-72.
140. Baratelli, F., et al., Pre-clinical characterization of GMP grade CCL21-gene modified dendritic cells for application in a phase I trial in non-small cell lung cancer. J Transl Med, 2008. 6: p. 38.
141. Hegmans, J. P., et al., Consolidative dendritic cell-based immunotherapy elicits cytotoxicity against malignant mesothelioma. Am J Respir Crit Care Med, 2010. 181 (12): p. 1383-90.
142. Urn, S. J., et al., Phase I study of autologous dendritic cell tumor vaccine in patients with non-small cell lung cancer. Lung Cancer, 2010. 70 (2): p. 188-94.
143. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10 (6): p. 983-91.
144. Perroud, M. W., Jr., et al., Mature autologous dendritic cell vaccines in advanced non-small cell lung cancer: a phase I pilot study. J Exp Clin Cancer Res, 2011. 30: p. 65.
145. Skachkova, O. V., et al., Immunological markers of anti-tumor dendritic cells vaccine efficiency in patients with non-small cell lung cancer. Exp Oncol, 2013. 35 (2): p. 109-13.
146. Hernando, J. J., et al., Vaccination with autologous tumour antigen pulsed dendritic cells in advanced gynaecological malignancies: clinical and immunological evaluation of a phase I trial. Cancer Immunol Immunother, 2002. 51 (1): p. 45-52.
147. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61 (3): p. 373-84.
148. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61 (5): p. 629-41.
149. Kandalaft, L. E., et al., A Phase I vaccine trial using dendritic cells pulsed with autologous oxidized lysate for recurrent ovarian cancer. J Transl Med, 2013. 11: p. 149.
150. Lepisto, A. J., et al., A phase I/II study of a MUC1 peptide pulsed autologous dendritic cell vaccine as adjuvant therapy in patients with resected pancreatic and biliary tumors. Cancer Ther, 2008. 6(B): p. 955-964.
151. Rong, Y., et al., A phase I pilot trial of MUC1-peptide-pulsed dendritic cells in the treatment of advanced pancreatic cancer. Clin Exp Med, 2012. 12 (3): p. 173-80.
152. Endo, H., et al., Phase I trial of preoperative intratumoral injection of immature dendritic cells and OK-432 for resectable pancreatic cancer patients. J Hepatobiliary Pancreat Sci, 2012. 19 (4): p. 465-75.
153. Jackson, L., et al., Adult mesenchymal stem cells: differentiation potential and therapeutic applications. J Postgrad Med, 2007. 53 (2): p. 121-7.
154. Pittenger, M. F., et al., Multilineage potential of adult human mesenchymal stem cells. Science, 1999. 284 (5411): p. 143-7.
155. Banas, A., et al., Rapid hepatic fate specification of adipose-derived stem cells and their therapeutic potential for liver failure. J Gastroenterol Hepatol, 2009. 24 (1): p. 70-7.
156. Lee, K. D., et al., In vitro hepatic differentiation of human mesenchymal stem cells. Hepatology, 2004. 40 (6): p. 1275-84.
157. Cho, K. A., et al., Mesenchymal stem cells showed the highest potential for the regeneration of injured liver tissue compared with other subpopulations of the bone marrow. Cell Biol Int, 2009. 33 (7): p. 772-7.
158. Hong, S. H., et al., In vitro differentiation of human umbilical cord blood-derived mesenchymal stem cells into hepatocyte-like cells. Biochem Biophys Res Commun, 2005. 330 (4): p. 1153-61.
159. Ishikawa, T., et al., Stem cells for hepatic regeneration: the role of adipose tissue derived mesenchymal stem cells. Curr Stem Cell Res Ther, 2010. 5 (2): p. 182-9.
160. Seo, M. J., et al., Differentiation of human adipose stromal cells into hepatic lineage in vitro and in vivo. Biochem Biophys Res Commun, 2005. 328 (1): p. 258-64.
161. Crisan, M., et al., A perivascular origin for mesenchymal stem cells in multiple human organs. Cell Stem Cell, 2008. 3 (3): p. 301-13.
162. Tavian, M. and B. Peault, Embryonic development of the human hematopoietic system. Int J Dev Biol, 2005. 49 (2-3): p. 243-50.
163. Peault, B., et al., Stem and progenitor cells in skeletal muscle development, maintenance, and therapy. Mol Ther, 2007. 15 (5): p. 867-77.
164. Aggarwal, S. and M. F. Pittenger, Human mesenchymal stem cells modulate allogeneic immune cell responses. Blood, 2005. 105 (4): p. 1815-22.
165. Caplan, A. I., Adult mesenchymal stem cells for tissue engineering versus regenerative medicine. J Cell Physiol, 2007. 213 (2): p. 341-7.
166. Chamberlain, G., et al., Concise review: mesenchymal stem cells: their phenotype, differentiation capacity, immunological features, and potential for homing. Stem Cells, 2007. 25 (11): p. 2739-49.
167. Banas, A., et al., IFATS collection: in vivo therapeutic potential of human adipose tissue mesenchymal stem cells after transplantation into mice with liver injury. Stem Cells, 2008. 26 (10): p. 2705-12.
168. Kharaziha, P., et al., Improvement of liver function in liver cirrhosis patients after autologous mesenchymal stem cell injection: a phase I-II clinical trial. Eur J Gastroenterol Hepatol, 2009. 21 (10): p. 1199-205.
169. Kuo, T. K., et al., Stem cell therapy for liver disease: parameters governing the success of using bone marrow mesenchymal stem cells. Gastroenterology, 2008. 134 (7): p. 2111-21, 2121 el-3.
170. Chang, Y. J., et al., Mesenchymal stem cells facilitate recovery from chemically induced liver damage and decrease liver fibrosis. Life Sci, 2009. 85 (13-14): p. 517-25.
171. Lu, L. L., et al., Isolation and characterization of human umbilical cord mesenchymal stem cells with hematopoiesis-supportive function and other potentials. Haematologica, 2006. 91 (8): p. 1017-26.
172. Mohamadnejad, M., et al., Phase 1 trial of autologous bone marrow mesenchymal stem cell transplantation in patients with decompensated liver cirrhosis. Arch Iran Med, 2007. 10 (4): p. 459-66.
173. Terai, S., et al., Improved liver function in patients with liver cirrhosis after autologous bone marrow cell infusion therapy. Stem Cells, 2006. 24 (10): p. 2292-8.
174. Chang, C. J., et al., Placenta-derived multipotent cells exhibit immunosuppressive properties that are enhanced in the presence of interferon-gamma. Stem Cells, 2006. 24 (11): p. 2466-77.
175. Iyer, S. S. and M. Rojas, Anti-inflammatory effects of mesenchymal stem cells: novel concept for future therapies. Expert Opin Biol Ther, 2008. 8 (5): p. 569-81.
176. Nauta, A. J. and W. E. Fibbe, Immunomodulatory properties of mesenchymal stromal cells. Blood, 2007. 110 (10): p. 3499-506.
177. Uccelli, A., V. Pistoia, and L. Moretta, Mesenchymal stem cells: a new strategy for immunosuppression? Trends Immunol, 2007. 28 (5): p. 219-26.
178. Wolbank, S., et al., Dose-dependent immunomodulatory effect of human stem cells from amniotic membrane: a comparison with human mesenchymal stem cells from adipose tissue. Tissue Eng, 2007. 13 (6): p. 1173-83.
179. Wolf, D. and A. M. Wolf, Mesenchymal stem cells as cellular immunosuppressants. Lancet, 2008. 371 (9624): p. 1553-4.
180. Shi, M., Z. W. Liu, and F. S. Wang, Immunomodulatory properties and therapeutic application of mesenchymal stem cells. Clin Exp Immunol, 2011. 164 (1): p. 1-8.
181. Sordi, V. and L. Piemonti, Therapeutic plasticity of stem cells and allograft tolerance. Cytotherapy, 2011. 13 (6): p. 647-60.
182. Popp, F. C., et al., Mesenchymal stem cells as immunomodulators after liver transplantation. Liver Transpl, 2009. 15 (10): p. 1192-8.
183. Friedenstein, A. J., R. K. Chailakhjan, and K. S. Lalykina, The development of fibroblast colonies in monolayer cultures of guinea-pig bone marrow and spleen cells. Cell Tissue Kinet, 1970. 3 (4): p. 393-403.
184. Friedenstein, A. J., et al., Stromal cells responsible for transferring the microenvironment of the hemopoietic tissues. Cloning in vitro and retransplantation in vivo. Transplantation, 1974. 17 (4): p. 331-40.
185. Caplan, A. I., Molecular and cellular differentiation of muscle, cartilage, and bone in the developing limb. Prog Clin Biol Res, 1986. 217B: p. 307-18.
186. Caplan, A. I., Mesenchymal stem cells. J Orthop Res, 1991. 9 (5): p. 641-50.
187. Kopen, G. C., D. J. Prockop, and D. G. Phinney, Marrow stromal cells migrate throughout forebrain and cerebellum, and they differentiate into astrocytes after injection into neonatal mouse brains. Proc Natl Acad Sci USA, 1999. 96 (19): p. 10711-6.
188. Petersen, B. E., et al., Bone marrow as a potential source of hepatic oval cells. Science, 1999. 284 (5417): p. 1168-70.
189. Teratani, T., et al., Direct hepatic fate specification from mouse embryonic stem cells. Hepatology, 2005. 41 (4): p. 836-46.
190. Le Blanc, K. and O. Ringden, Immunomodulation by mesenchymal stem cells and clinical experience. J Intern Med, 2007. 262 (5): p. 509-25.
191. Keyser, K. A., K. E. Beagles, and H. P. Kiem, Comparison of mesenchymal stem cells from different tissues to suppress T-cell activation. Cell Transplant, 2007. 16 (5): p. 555-62.
192. Le Blanc, K., et al., Mesenchymal stem cells for treatment of steroid-resistant, severe, acute graft-versus-host disease: a phase II study. Lancet, 2008. 371 (9624): p. 1579-86.
193. Ning, H., et al., The correlation between cotransplantation of mesenchymal stem cells and higher recurrence rate in hematologic malignancy patients: outcome of a pilot clinical study. Leukemia, 2008. 22 (3): p. 593-9.
194. Ball, L., et al., Third party mesenchymal stromal cell infusions fail to induce tissue repair despite successful control of severe grade IV acute graft-versus-host disease in a child with juvenile myelo-monocytic leukemia. Leukemia, 2008. 22 (6): p. 1256-7.
195. Ringden, O., et al., Mesenchymal stem cells for treatment of therapy-resistant graft-versus-host disease. Transplantation, 2006. 81 (10): p. 1390-7.
196. Le Blanc, K., et al., Treatment of severe acute graft-versus-host disease with third party haploidentical mesenchymal stem cells. Lancet, 2004. 363 (9419): p. 1439-41.
197. Muller, I., et al., Application of multipotent mesenchymal stromal cells in pediatric patients following allogeneic stem cell transplantation. Blood Cells Mol Dis, 2008. 40 (1): p. 25-32.
198. Horwitz, E. M., et al., Isolated allogeneic bone marrow-derived mesenchymal cells engraft and stimulate growth in children with osteo genesis imperfecta: Implications for cell therapy of bone. Proc Natl Acad Sci USA, 2002. 99 (13): p. 8932-7.
199. Koc, O. N., et al., Allogeneic mesenchymal stem cell infusion for treatment of metachromatic leukodystrophy (MLD) and Hurler syndrome (MPS-IH). Bone Marrow Transplant, 2002. 30 (4): p. 215-22.
200. Le Blanc, K., et al., Transplantation of mesenchymal stem cells to enhance engraftment of hematopoietic stem cells. Leukemia, 2007. 21 (8): p. 1733-8.
201. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11 (5): p. 389-98.
202. Ball, L. M., et al., Cotransplantation of ex vivo expanded mesenchymal stem cells accelerates lymphocyte recovery and may reduce the risk of graft failure in haploidentical hematopoietic stem-cell transplantation. Blood, 2007. 110 (7): p. 2764-7.
203. http://www.osiristx.com/pdf/PR %2039%2025Mar07%20Provacel %20Positive %2 OResults.pdf.
204. Kurtzberg, J., et al., Allogeneic human mesenchymal stem cell therapy (remestemcel-L, Prochymal) as a rescue agent for severe refractory acute graft-versus-host disease in pediatric patients. Biol Blood Marrow Transplant, 2014. 20 (2): p. 229-35.
205. Kellathur, S. N. and H. X. Lou, Cell and tissue therapy regulation: worldwide status and harmonization. Biologicals, 2012. 40 (3): p. 222-4.
206. Ahmadi, M., et al., Bone marrow mesenchymal stem cells and their conditioned media could potentially ameliorate ovalbumin-induced asthmatic changes. Biomed Pharmacother, 2016. 85: p. 28-40.
207. Nemunaitis, J., et al., Human marrow stromal cells: response to interleukin-6 (IL-6) and control of IL-6 expression. Blood, 1989. 74 (6): p. 1929-35.
208. Sadovnikova, E. Y., et al., Induction of hematopoietic microenvironment by the extracellular matrix from long-term bone marrow cultures. Ann Hematol, 1991. 62 (5): p. 160-4.
209. Lazarus, H. M., et al., Ex vivo expansion and subsequent infusion of human bone marrow-derived stromal progenitor cells (mesenchymal progenitor cells): implications for therapeutic use. Bone Marrow Transplant, 1995. 16 (4): p. 557-64.
210. Yoo, J. U., et al., The chondrogenic potential of human bone-marrow-derived mesenchymal progenitor cells. J Bone Joint Surg Am, 1998. 80 (12): p. 1745-57.
211. Fleming, J. E., Jr., et al., Monoclonal antibody against adult marrow-derived mesenchymal stem cells recognizes developing vasculature in embryonic human skin. Dev Dyn, 1998. 212 (1): p. 119-32.
212. Ghilzon, R., C. A. McCulloch, and R. Zohar, Stromal mesenchymal progenitor cells. Leuk Lymphoma, 1999. 32 (3-4): p. 211-21.
213. De Cesaris, V., et al., Isolation, proliferation and characterization of endometrial canine stem cells. Reprod Domest Anim, 2016.
214. Van Pham, P., et al., Isolation and proliferation of umbilical cord tissue derived mesenchymal stem cells for clinical applications. Cell Tissue Bank, 2016. 17 (2): p. 289-302.
215. Zhao, G., et al., Large-scale expansion of Wharton's jelly-derived mesenchymal stem cells on gelatin microbeads, with retention of self-renewal and multipotency characteristics and the capacity for enhancing skin wound healing. Stem Cell Res Ther, 2015. 6: p. 38.
216. Huang, P., et al., Differentiation of human umbilical cord Wharton's jelly-derived mesenchymal stem cells into germ-like cells in vitro. J Cell Biochem, 2010. 109 (4): p. 747-54.
217. Wang, S. J., et al., Chondrogenic Potential of Peripheral Blood Derived Mesenchymal Stem Cells Seeded on Demineralized Cancellous Bone Scaffolds. Sci Rep, 2016. 6: p. 36400.
218. Fazeli, Z., M. D. Omrani, and S. M. Ghaderian, CD29/CD184 expression analysis provides a signature for identification of neuronal like cells differentiated from PBMSCs. Neurosci Lett, 2016. 630: p. 189-93.
219. Wu, G., et al., Osteogenesis of peripheral blood mesenchymal stem cells in self assembling peptide nanofiber for healing critical size calvarial bony defect. Sci Rep, 2015. 5: p. 16681.
220. Shaer, A., et al., Isolation and characterization of Human Mesenchymal Stromal Cells Derived from Placental Decidua Basalis; Umbilical cord Wharton's Jelly and Amniotic Membrane. Pak J Med Sci, 2014. 30 (5): p. 1022-6.
221. Fu, W. L., C. Y. Zhou, and J. K. Yu, A new source of mesenchymal stem cells for articular cartilage repair: MSCs derived from mobilized peripheral blood share similar biological characteristics in vitro and chondrogenesis in vivo as MSCs from bone marrow in a rabbit model. Am J Sports Med, 2014. 42 (3): p. 592-601.
222. El-Badawy, A., et al., Adipose Stem Cells Display Higher Regenerative Capacities and More Adaptable Electro-Kinetic Properties Compared to Bone Marrow-Derived Mesenchymal Stromal Cells. Sci Rep, 2016. 6: p. 37801.
223. Plock, J. A., et al., The Influence of Timing and Frequency of Adipose-Derived Mesenchymal Stem Cell Therapy on Immunomodulation Outcomes After Vascularized Composite Allotransplantation. Transplantation, 2017. 101 (1): p. el-ell.

1. A method of treating a degenerative condition comprising: a) extracting a cellular population resembling gamma delta T cells; b) expanding said population ex vivo; c) endowing said cell with one or more regenerative activities; and d) administering said cell in a patient in need of therapy.

2. The method of claim 1, wherein said T cells express CD3.

3. The method of claim 1, wherein said T cells express CD6.

4. The method of claim 1, wherein said T cells express CD27.

5. The method of claim 1, wherein said T cells express il-2 receptor.

6. The method of claim 1, wherein said T cells express CD25.

7. The method of claim 1, wherein said T cells proliferate in response to IL-2.

8. The method of claim 1, wherein said T cells proliferate in response to IL-7.

9. The method of claim 1, wherein said T cells express the gamma delta T cell receptor.

10. The method of claim 1, wherein said T cells do not express the alpha beta T cell receptor.

11. The method of claim 1, wherein said T cells recognized conserved antigens.

12. The method of claim 1, wherein said T cells are less immunogenic as compared to conventional T cells.

13. The method of claim 12, wherein said conventional T cells are CD4 alpha beta T cells.

14. The method of claim 12, wherein said conventional T cells are CD8 alpha beta T cells.

15. The method of claim 12, wherein said immunogenicity means ability to stimulate proliferation of allogeneic T cells.

16. The method of claim 12, wherein said immunogenicity means ability to stimulate cytotoxicity of allogeneic T cells.

17. The method of claim 12, wherein said immunogenicity means ability to stimulate NF-kappa B activation in allogeneic T cells.

18. The method of claim 12, wherein said immunogenicity means ability to stimulate cytokine secretion of allogeneic T cells.

19. The method of claim 12, wherein said cytokine is IL-2.

20. The method of claim 1, wherein said regeneration is stimulation of angiogenesis.
